(19) United States
(12) Patent Application Publication
(10) Pub. No.: US 2019/0046436 A1
(43) Pub. Date: Feb. 14, 2019

(54) NICOTINE FORMULATION AND AEROSOLS

(71) Applicant: NICOGEN LTD., Migdal Haemek (IL)

(72) Inventor: Miron HAZANI, Haifa (IL)

(21) Appl. No.: 16/078,294

(22) PCT Filed: Feb. 28, 2017
(86) PCT No.: PCT/IL2017/050252

§ 371 Date: Aug. 21, 2018

Related U.S. Application Data
(60) Provisional application No. 62/300,947, filed on Feb. 29, 2016.
(60) Provisional application No. 62/439,911, filed on Dec. 29, 2016.

(51) Int. Cl. A61K 9/00; A24F 47/00; A24B 15/16; A61K 31/465; A61K 9/08; A61K 47/12; A61K 47/26; A61K 47/22; A61K 47/18

(52) U.S. Cl. CPC A61K 9/0078; A24F 47/008; A24B 15/167; A61K 31/465; A61K 47/183; A61K 47/12; A61K 47/26; A61K 47/22; A61K 9/08

(57) ABSTRACT

The present disclosure generally relates to nicotine formulations, nebulizer systems comprising same and uses thereof via inhalation.

### TECHNICAL FIELD

The present disclosure generally relates to nicotine formulations, nebulizer systems encompassing same and uses thereof via inhalation.

### BACKGROUND

Nebulizers are commonly used for delivering aerosol medication to patients via the respiratory system. Desirably, for efficient delivery of medication, the droplet diameter of the aerosol should be sufficiently small so as to reach the lungs of the patient without being obstructed by objects or organs (such as, the inner surface of the nozzle in the nebulizer and the mouth cavity perimeters) and large enough so as to remain in the lungs during exhalation.

Nicotine is available in several forms for use as an alternative to tobacco addiction without exposure to the carcinogens of the tobacco products

U.S. Pat. No. 4,953,572 discloses a method and a composition for aiding in the reduction of the incidence of tobacco smoking, the method includes administering an aerosol spray containing low concentrations of nicotine, about 0.005 mg to about 0.03 mg per inhalation, with a droplet size is 1 to 10 microns, and a pH of at least 7.

US Publication No. 2008/0066741 discloses systems and methods for delivery of a drug to the respiratory system of a patient, where the drug is supplied in purified air at a positive pressure relative to atmospheric pressure and where the drug may be nicotine.

US Publication No. 2014/0261474 discloses a system that delivers nicotine to target the regions of the respiratory tract to achieve the maximum impact on craving with a minimum number of unwanted sensations and a method that relates to pulmonary administration of nicotine from nicotine inhalation systems that target delivery to the deep lung, minimizing deposition in the upper and central airways.

WO/2016/059630 to the inventor of the present invention discloses a nebulizer comprising a porous medium configured to produce aerosols, a displaceable wetting mechanism configured to spread a liquid over the porous medium thereby to wet the porous medium and a gas channel configured to introduce pressure gradient to the porous medium.

There is an unmet need for a nicotine formulation which, upon inhalation a dose thereof, would be bioequivalent to cigarettes, including in terms of pharmacokinetics and overall user's satisfaction.

### SUMMARY

The following embodiments and aspects thereof are described and illustrated in conjunction with formulations, nebulizer systems and methods which are meant to be exemplary and illustrative, not limiting in scope. In various embodiments, one or more of the above-described problems have been reduced or eliminated, while other embodiments are directed to other advantages or improvements.

According to some embodiments, there are provided herein formulations, devices, systems and methods for generating aerosol comprising pure nicotine at pH below 6, and for delivery of said aerosol using a porous medium and a displaceable spreading mechanism or liquid absorbing material. Advantageously, the nicotine formulations disclosed herein, when delivered via inhalation, exhibit pharmacokinetics similar to cigarettes, thereby providing an efficient substitute for smokers, which is safe and is devoid of smoke. Specifically, the nicotine formulations disclosed herein, when delivered via inhalation, exhibit an AUC within the range of 80-100% compared to a cigarette and Tmax of 3 to 5 minutes. Moreover, the nicotine formulation disclosed herein, when delivered via inhalation, provide Cmax which is highly proportional to the inhaled dose. Thus, the formulations disclosed herein are suitable for handling smoking withdrawal, particularly in patients suffering from, or susceptible to, a disease or disorder related to the respiratory system.

According to some embodiments, there is provided a nicotine formulation comprising nicotine and a buffer, wherein the formulation is having a pH below 6, and wherein the nicotine is having a purity of at least 95%.

According to some embodiments, the nicotine formulation is an aqueous nicotine formulation. According to some embodiments, the buffer is an aqueous buffer.

According to some embodiments, the buffer is selected from citrate buffers, phosphate buffers and a combination thereof. According to some embodiments, the buffer comprises a citrate buffer.

According to some embodiments, the percentage of nicotine based on the total mass of the nicotine formulation is within the range of 0.1 to 10%. According to some embodiments, the percentage of nicotine based on the total mass of the nicotine formulation is within the range of 0.5 to 8%. According to some embodiments, the percentage of nicotine based on the total mass of the nicotine formulation is within the range of 0.7 to 6%. According to some embodiments, the percentage of nicotine based on the total mass of the nicotine formulation is within the range of 1 to 4%.

According to some embodiments, the concentration of nicotine in the formulation is within the range of 2 to 200 mg/ml. According to some embodiments, the concentration of nicotine in the formulation is within the range of 3 to 90 mg/ml. According to some embodiments, the concentration of nicotine in the formulation is within the range of 4 to 75 mg/ml. According to some embodiments, the concentration of nicotine in the formulation is within the range of 7.5 to 60 mg/ml. According to some embodiments, the concentration of nicotine in the formulation is within the range of 10 to 40 mg/ml.

According to some embodiments, the nicotine formulation is having a pH below 6. According to some embodiments, the nicotine formulation is having a pH within the range of 3.0 to 5.0. According to some embodiments, the nicotine formulation is having a pH within the range of 3.5 to 4.5. According to some embodiments, the nicotine formulation is having a pH of about 4.

According to some embodiments, the nicotine is the sole active ingredient in the nicotine formulation.

According to some embodiments, the formulation further comprises at least one anti-coughing agent.

According to some embodiments, the formulation further comprises at least one preservative.

According to some embodiments, the nicotine formulation is an aerosol. According to some embodiments, the nicotine formulation is an aerosol devoid of propellants.

According to some embodiments, there is provided an aerosol comprising a nicotine formulation, said aerosol comprising droplets having an MMAD of at most 10 microns, wherein the nicotine formulation comprises nicotine and a buffer, wherein the formulation is having a pH below 6, and wherein the nicotine is having a purity of at least 95%.

According to some embodiments, the nicotine formulation is an aqueous nicotine formulation. According to some embodiments, the buffer is an aqueous buffer.

According to some embodiments, the buffer is selected from citrate buffers phosphate buffers and a combination thereof. According to some embodiments, the buffer comprises a citrate buffer.

According to some embodiments, the nicotine is the only active ingredient in the nicotine formulation.

According to some embodiments, the aerosol comprises droplets having an MMAD within the range of 0.3 to 7 microns.

According to some embodiments, there is provided use of the nicotine formulation disclosed herein for the treatment of a disease or disorder.

According to some embodiments, there is provided a nebulizer comprising a porous medium configured to produce aerosols, a displaceable wetting mechanism configured to spread a liquid over the porous medium thereby to wet the porous medium and a gas channel configured to introduce pressure gradient to the porous medium, wherein the liquid comprises a nicotine formulation, comprising nicotine and a buffer, wherein the formulation is having a pH below 6.

According to some embodiments, the nicotine is the sole active ingredient in the nicotine formulation. According to some embodiments, the nicotine is having a purity of at least 95%.

According to some embodiments, the percentage of nicotine based on the total mass of the formulation is within the range of 0.1 to 10%.

According to some embodiments, the nicotine formulation is having a pH within the range of 3.5 to 4.5. According to some embodiments, the nicotine formulation is having a pH of about 4.

According to some embodiments, the nicotine formulation is an aqueous nicotine formulation. According to some embodiments, the buffer is an aqueous buffer.

According to some embodiments, the buffer is selected from citrate buffers, phosphate buffers and combinations thereof. According to some embodiments, the buffer comprises a citrate buffer.

According to some embodiments, the displaceable wetting mechanism comprises a rotatable elongated member.

According to some embodiments, the rotatable elongated member is configured to move across the surface of the porous medium, thereby to homogeneously or semi-homogeneously spread the liquid over the surface.

According to some embodiments, the rotatable elongated member is axially movable.

According to some embodiments, the rotatable elongated member is movable to cover approximately all the surface of the porous medium.

According to some embodiments, the elongated, member is at least partially covered with polytetrafluoroethylene (PTFE), commercially knowns as Teflon®, or any other appropriate coating materials.

According to some embodiments, the elongated member is an elongated tubular member. According to some embodiments, the elongated member is movable by an actuator, mechanically connected thereto. According to some embodiments, the elongated member is movable by the air-flow within the nebulizer and/or through the porous material.

According to some embodiments, the elongated member is a roller. According to some embodiments, the elongated member is a smearing device. According to some embodiments, the elongated member is a spreading device. According to some embodiments, the elongated member is configured to force at least portions of the liquid to at least some of the pores of the porous medium.

According to some embodiments, the nebulizer further comprises a spacer configured to elevate said displaceable wetting mechanism from the surface of said porous medium. According to some embodiments, said spacer is integrally formed with said displaceable wetting mechanism. According to some embodiments, said spacer comprises a protrusion in said displaceable wetting mechanism. According to some embodiments, said spacer is configured to be placed between said displaceable wetting mechanism and the surface of said porous medium. According to some embodiments, said pacer comprises a ring-shaped configured to facilitate low-friction displacement of said displaceable wetting mechanism.

According to some embodiments, the nebulizer further comprises a liquid deploying mechanism configured to controllably deploy the liquid on the surface of said porous medium for being spread by said displaceable wetting mechanism. According to some embodiments, said liquid deploying mechanism comprises a conduit. According to some embodiments, said conduit has a receiving end, configured to obtain the liquid from a liquid source, and a deploying end, configured to deploy the liquid on the surface of said porous medium. According to some embodiments, said deploying end of said conduit is flexible and configured to flexibly move by the displacement of said displaceable wetting mechanism, thereby deploy the liquid at more than one location on the surface of said porous medium.

According to some embodiments, the nebulizer further comprises an opening configured to deliver the aerosols to a respiratory system of a subject.

According to some embodiments, the displaceable wetting mechanism further comprises an actuator configured to displace or induce the displacement of the rotatable elongated member.

The term “displacement” as used herein may be interchangeable with any one or more of the terms movement, movement across. This term may refer to the motion of the wetting mechanism across, or along, at least one surface of the porous medium.

According to some embodiments, the rotatable elongated member comprises a first magnet, and the actuator comprises a second magnet, magnetically associated with said first magnet, such that by moving the second magnet displacement of the rotatable elongated member is induced.

According to some embodiments, the said first and/or second magnet comprise a plurality of magnets. According to some embodiments, one or more of the plurality of magnets comprises an electromagnet.

According to some embodiments, the actuator comprises a motor configured to displace the rotatable elongated member.

According to some embodiments, there is provided use of the aforementioned nebulizer for the treatment of a disease or disorder.

According to some embodiments, there is provided a nebulizer comprising a porous medium configured to produce aerosols, a liquid absorbing material configured to absorb a liquid, a wetting mechanism configured to press the liquid absorbing material against the porous medium, thereby to wet the porous medium with the liquid absorbed in the liquid absorbing material and a gas channel configured to introduce pressure gradient to the porous medium, wherein the liquid comprises a nicotine formulation, comprising nicotine and a buffer, wherein the formulation is having a pH below 6. According to some embodiments, the nicotine formulation is an aqueous nicotine formulation. According to some embodiments, the buffer is an aqueous buffer.

According to some embodiments, the nicotine formulation is consisting of nicotine and a buffer.

According to some embodiments, the buffer is selected from citrate buffers, phosphate buffers and combinations thereof. According to some embodiments, the buffer comprises a citrate buffer.

According to some embodiments, the nicotine is the only active ingredient in the nicotine formulation. According to some embodiments, the nicotine is having a purity of at least 95%. According to some embodiments, the percentage of nicotine is within the range of 0.1 to 10%.

According to some embodiments, the nicotine formulation is having a pH within the range of 3.5 to 4.5. According to some embodiments, the nicotine formulation is having a pH of about 4.

According to some embodiments, the liquid absorbing material is selected from a sponge, a tissue and foam.

According to some embodiments, the liquid absorbing material comprises a sponge.

According to some embodiments, the sponge comprises an open cell foam and/or a closed cell foam.

According to some embodiments, the liquid absorbing material is configured to act as an impactor for aerosols produced by the porous medium.

According to some embodiments, the liquid absorbing material is configured to act as a filter for aerosols produced by the porous medium.

According to some embodiments, the liquid absorbing material comprises a composition comprising the nicotine formulation, said composition is at least partially absorbed within the liquid absorbing material.

According to some embodiments, the composition comprising the nicotine formulation is absorbed within the liquid absorbing material.

According to some embodiments, the nebulizer further comprising a first container, configured to contain liquid to be delivered to the liquid absorbing material.

According to some embodiments, the nebulizer further comprising a second container configured to contain at least one composition comprising the nicotine formulation.

According to some embodiments, the liquid comprises water.

According to some embodiments, the gas channel is connected to a gas source.

According to some embodiments, there is provided use of the aforementioned nebulizer and the nicotine formulation included therein for the treatment of a disease or disorder.

According to some embodiments, there is provided a nebulizer cartridge comprising a porous medium, wherein the porous medium comprises a plurality of pores, and wherein at least some of said plurality of pores comprise liquid comprising a nicotine formulation comprising nicotine and a buffer, wherein the nicotine formulation is having a pH below 6.

According to some embodiments, the nicotine is the only active ingredient in the nicotine formula.

According to some embodiments, there is provided a nebulizer cartridge comprising a porous medium and a displaceable wetting mechanism configured to spread a liquid over the porous medium thereby to wet the porous medium, wherein the porous medium comprises a plurality of pores, and wherein at least some of said plurality of pores comprise liquid comprising a nicotine formulation comprising nicotine and a buffer, wherein the nicotine formulation is having a pH below 6.

According to some embodiments, the buffer is a citrate buffer.

According to some embodiments, the nicotine is having a purity of at least 95%.

According to some embodiments, the percentage of nicotine is within the range of 0.5 to 2%.

According to some embodiments, the nicotine formulation is having a pH within the range of 3.5 to 4.5. According to some embodiments, the nicotine formulation is having a pH of about 4.

According to some embodiments, the displaceable wetting mechanism comprises a rotatable elongated member.

According to some embodiments, the rotatable elongated member further comprises an actuator configured to displace or induce the displacement of the rotatable elongated member.

According to some embodiments, the rotatable elongated member comprises a first magnet, and the actuator comprises a second magnet, magnetically associated with said first magnet, such that by moving the second magnet displacement of the rotatable elongated member is induced.

According to some embodiments, the nebulizer cartridge is configured to be inserted to a nebulizer main body.

According to some embodiments, the nebulizer main body comprises an opening configured to deliver aerosols.

According to some embodiments, there is provided use of the aforementioned nebulizer cartridge, and the nicotine formulation included therein, for the treatment of a disease or disorder.

According to some embodiments, there is provided a nebulizer cartridge comprising a porous medium and a liquid absorbing material, configured to be pressed against the porous medium thereby producing aerosols, wherein the liquid absorbing material comprises a liquid at least partially absorbed therein, said liquid comprises a nicotine formulation comprising nicotine and a buffer, wherein the formulation is having a pH below 6.

The term “partially absorbed therein” as used herein refers to the percentage of liquid absorbed in the pores of the porous material, wherein 0% refers to a porous material where all of its pores are vacant of liquid. Thus, partially absorbed therein may refer to a porous material wherein at least 0.005% of the pores contain liquid, or wherein the overall contents of the vacant space within the porous material occupied with liquid is 0.005%. According to some embodiments, partially absorbed therein refers to at least 0.001% liquid contents within the porous material. According to some embodiments, partially absorbed therein refers to at least 0.05% liquid contents within the porous material. According to some embodiments, partially absorbed therein refers to at least 0.01% liquid contents within the porous material. According to some embodiments, partially absorbed therein refers to at least 0.5% liquid contents within the porous material. According to some embodiments, partially absorbed therein refers to at least 0.1% liquid contents within the porous material. According to some embodiments, partially absorbed therein refers to at least 1% liquid contents within the porous material. According to some embodiments, partially absorbed therein refers to at least 5% liquid contents within the porous material. According to some embodiments, partially absorbed therein refers to at least 10% liquid contents within the porous material. According to some embodiments, partially absorbed therein refers to at least 20% liquid contents within the porous material. According to some embodiments, partially absorbed therein refers to at least 30% liquid contents within the porous material. According to some embodiments, partially absorbed therein refers to at least 40% liquid contents within the porous material. According to some embodiments, partially absorbed therein refers to at least 50% liquid contents within the porous material.

According to some embodiments, partially absorbed therein may refer to the content of liquid within the volume of pores located on the surface and in the immediate vicinity of the surface (sub surface) of a porous medium. According to some embodiments, the volume of the sub-surface may extend from the surface to a depth of about 50 micron from the surface.

According to some embodiments, partially absorbed therein refers to a porous material wherein at least 0.5% of the surface and sub-surface pores contain liquid. According to some embodiments, partially absorbed therein refers to at least 1% liquid contents within the surface and sub-surface pores. According to some embodiments, partially absorbed therein refers to at least 10% liquid contents within the surface and sub-surface pores. According to some embodiments, partially absorbed therein refers to at least 20% liquid contents within the surface and sub-surface pores. According to some embodiments, partially absorbed therein refers to at least 30% liquid contents within the surface and sub-surface pores. According to some embodiments, partially absorbed therein refers to at least 40% liquid contents within the surface and sub-surface pores. According to some embodiments, partially absorbed therein refers to at least 50% liquid contents within the surface and sub-surface pores. According to some embodiments, partially absorbed therein refers to at least 60% liquid contents within the surface and sub-surface pores.

According to some embodiments, the nicotine is the only active ingredient in the nicotine formulation. According to some embodiments, the nicotine is having a purity of at least 95%.

According to some embodiments, the percentage of nicotine is within the range of 0.3 to 10%.

According to some embodiments, the nicotine formulation is having a pH within the range of 3.5 to 4.5. According to some embodiments, the nicotine formulation is having a pH of about 4.

According to some embodiments, the nicotine formulation is an aqueous nicotine formulation. According to some embodiments, the buffer is an aqueous buffer.

According to some embodiments, the buffer is selected from citrate buffers and phosphate buffers. According to some embodiments, the buffer is a citrate buffer.

According to some embodiments, the liquid absorbing material is selected from a sponge, a tissue and foam.

According to some embodiments, the liquid absorbing material comprises a sponge.

The term “sponge” as used herein refers to an absorbing, porous and/or fibrous, natural or synthetic material. Typically, sponges are made of wettable cellular materials, such as cellulose, polyurethane, polyolefins and the like.

A porous medium is understood to be a two-phase product with voids and solid portions. Generally, in an open cell sponge the voids are interconnected, and the solid portions, which define the voids, are also interconnected. As a result, such structures have a plurality of pores where inner surfaces of individual pores are accessible from neighboring pores. In contrast, in closed cell sponges individual pores are separate and self-contained.

According to some embodiments, the sponge comprises an open cell foam.

According to some embodiments, the sponge comprises a closed cell foam. According to some embodiments, the sponge comprises an open cell foam and/or a closed cell foam.

According to some embodiments, the sponge is made of a material selected from the group consisting of melamine foam, melamine-formaldehyde resin, polyurethane foam, urea-formaldehyde resin, polyether foam, polyester foam, unsaturated polyester resin, epoxy resin, phenol-formaldehyde resin, polyvinyl acetal foam, polyvinyl acetate foam, vinyl foam, acrylic foam, polystyrene foam, nylon foam, cyanoacrylate foam, silicone foam, polyethylene foam, polyvinyl butyral foam, polyvinyl neoprene foam, polyvinyl alcohol foam, foam, polyisoyanate foam, cellulose, cotton, paper, starch, felt, polyvinyl alcohol and any combination thereof. Each possibility is a separate embodiment of the invention.

As used herein, the term “porous” refers to any material that includes one or more of pores, cracks, fissures, vugs and voids extending into the material from external surfaces thereof. Further, the term “pore” includes and encompasses cracks, fissures, vugs and voids. Porous materials may include, for example, sponge, felt, paper, sand, cotton-wool silica, concrete, alumino-silicates, metals, minerals, polymers, ceramics, composites, asphalt, brick and mortar. Typically, the pores allow a fluid flow therethrough, including liquid materials, such as aqueous solutions.

According to some embodiments, the nebulizer cartridge is further comprising a container configured to contain the liquid to be delivered to the liquid absorbing material.

According to some embodiments, the nebulizer cartridge is configured to be inserted to a nebulizer main body.

According to some embodiments, there is provided use of the aforementioned nebulizer cartridge and the nicotine formulation included therein for the treatment of a disease or disorder.

According to some embodiments, there is provided the nicotine formulation for use in the treatment of a disease or disorder. The formulation may be included in the nebulizers and/or within the nebulizer cartridges disclosed herein.

According to some embodiments, there is provided a method for treating a disease or disorder in a subject in need thereof comprising administering, via inhalation, to a subject in need thereof an aerosol comprising a nicotine formulation, comprising nicotine and a buffer, having a pH below 6.

According to some embodiments, the nicotine is the only active ingredient in the nicotine formulation. According to some embodiments, the nicotine is having a purity of at least 95%.

According to some embodiments, the buffer is a citrate buffer.

According to some embodiments, the disorder is nicotine withdrawal syndrome.

According to some embodiments, the subject is having a disease or disorder related to the respiratory system.

According to some embodiments, the disease is asthma.

According to some embodiments, there is provided a method for producing aerosols comprising nicotine, the method comprises:

providing a nebulizer comprising a porous medium configured to produce aerosols, a displaceable wetting mechanism configured to spread the liquid over the porous medium thereby to wet the porous medium and a gas channel, wherein said porous medium is having two sides, a first side facing the displaceable wetting mechanism; providing a liquid comprising a nicotine formulation comprising nicotine and a buffer, wherein the nicotine formulation is having a pH below 6; operating the displaceable wetting mechanism thereby spreading the liquid onto said first side of the porous medium; and connecting the gas channel to a pressure source and introducing pressure gradient to the porous medium thereby producing aerosol at the first side of the porous medium, the aerosol comprises droplets of the liquid.

According to some embodiments, the nicotine is the only active ingredient in the nicotine formulation. According to some embodiments, the nicotine is having a purity of at least 95%. According to some embodiments, the buffer is an aqueous citrate buffer.

According to some embodiments, there is provided a method for producing nicotine aerosols, the method comprises: providing a nebulizer comprising a porous medium configured to produce aerosols, a liquid absorbing material configured to absorb a liquid, a wetting mechanism configured to press the liquid absorbing material against the porous medium, and a gas channel configured to introduce pressure gradient to the porous medium, wherein the porous medium is having two sides wherein a first side is facing the liquid absorbing material; providing liquid comprising a nicotine formulation comprising nicotine and a buffer, wherein the nicotine formulation is having a pH below 6; wetting the liquid absorbing material with the liquid; pressing the liquid absorbing material against the porous medium; and introducing pressure gradient to the porous medium thereby producing aerosol at the first side of the porous medium, the aerosol comprises droplets of the liquid.

According to some embodiments, the nicotine is the only active ingredient in the nicotine formulation. According to some embodiments, the nicotine is having a purity of at least 95%.

According to some embodiments, the method further comprises delivering the nicotine aerosols to a respiratory system of a subject in need thereof.

According to some embodiments, the method further comprises iterating the following steps at least one more time: pressing the liquid absorbing material against the porous medium, introducing pressure gradient to the porous medium and producing aerosol at the first side of the porous medium, the aerosol comprises droplets of the liquid.

According to some embodiments, pressing comprises applying a pressing force that varies over iterations.

According to some embodiments, the method further comprises providing a cleansing liquid and iterating the following steps with the cleansing liquid: wetting the liquid absorbing material with the liquid, pressing the liquid absorbing material against the porous medium, introducing pressure gradient to the porous medium and producing aerosol at the first side of the porous medium, the aerosol comprises droplets of the liquid.

According to some embodiments, the porous medium is rigid. According to some embodiments, the porous medium is made of metal. According to some embodiments, the porous medium has two flat sides, which remain flat when liquid is pressed therethrough. According to some embodiments, the porous medium is rigid where liquid is absorbed, or partially absorbed, therein.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 shows Mass Distribution on Impactor parts in an aerosol provided from a nebulizer according to the present invention and a formulation comprising 10 mg/ml nicotine in a citrate buffer adjusted to pH=4.

FIG. 2 shows cumulative Mass Distribution in an aerosol provided from a nebulizer according to the present invention with a formulation comprising 10 mg/ml nicotine in a citrate buffer adjusted to pH=4.

FIG. 3 schematically illustrates an exploded view of components within a nebulizer with a porous medium, according to some embodiments.

FIG. 4 schematically illustrates a perspective view of components within a nebulizer with a porous medium, according to some embodiments.

FIG. 5A shows average plasma nicotine concentrations versus time taken before and after inhalations of nicotine formulations at doses of 0.22 mg (squares; Day 1), 0.45 mg (diamonds; Day 2), 0.67 mg (circles; Day 3) and before and after smoking a cigarette (triangle; Day 4).

FIG. 5B is a portion of FIG. 5A, showing the 4th inhalation of each day.

FIG. 5C is a portion of FIG. 5A, showing up to 60 minutes post 4th inhalation each day.

FIG. 5D is a mathematical best fit of the values presented in FIG. 5B.

FIG. 6 shows Mass Distribution on Impactor parts in aerosols provided from a nebulizer as disclosed herein with four nicotine formulations comprising 10 mg/ml (squares), 20 mg/ml (circles), 30 mg/ml (diamonds) and 40 (triangles) mg/ml nicotine in a citrate buffer adjusted to pH=4.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

According to some embodiments, there is provided a nicotine formulation comprising nicotine and a buffer, having a pH below 6, wherein the nicotine is having a purity of at least 95%.

As used herein the term “purity” and “pure” relate to the chemical purity of a compound which may contain other chemical compounds as impurities wherein the particular compound is present in an amount of at least about 90%, preferably at least about 95%, more preferably at least about 99%, most preferably at least about 99.5% by weight. Typically, the purity can be measured by HPLC. Specifically, impurities commonly present in compositions of nicotine include its related oxidation side products, such as the dehydrogenated myosmine or the oxygenated cotinine and nicotine-N-oxide. Therefore, the term “nicotine having a purity of at least 95%” is meant to describe a composition in which the nicotine related impurities are present in a weight amount, which constitutes not more than 5% relative to the weight of the nicotine in the composition.

As used herein the term “formulation” generally refers to any mixture, solution, suspension or the like that contains an active ingredient, such as nicotine, and, optionally, a carrier and has physical properties such that when the formulation is moved through the respirator device as described herein, the formulation is in a form that is delivered/inhaled/blown by positive pressure into the lungs of a patient. The carrier may be any pharmaceutically acceptable flowable agent that is compatible for delivery with the active agent.

The term “buffer” is well known as a general description of a solution containing either a weak acid and its salt or a weak base and its salt, which is resistant to changes in pH. For example, the term “citrate buffer” is intended to describe a solution comprising citric acid and deprotonated citrate anion, such as, but not limited to, sodium citrate.

According to some embodiments the formulation includes diluted pure nicotine solutions containing 1 to 200 mg/ml, 2 to 150 mg/ml, 3 to 100 mg/ml, 4 to 80 mg/ml, 10 to 40 mg/ml, about 10 mg/ml, about 20 mg/ml, about 30 mg/ml or about 40 mg/ml nicotine.

According to some embodiments, the percentage of nicotine in the nicotine formulation is within the range of 0.1 to 10%. According to some embodiments, the percentage of nicotine in the nicotine formulation is within the range of 0.3 to 8%. According to some embodiments, the percentage of nicotine in the nicotine formulation is within the range of 0.5 to 6%. According to some embodiments, the percentage of nicotine in the nicotine formulation is within the range of 1 to 4%. According to some embodiments, the percentage of nicotine in the nicotine formulation is about 1%. According to some embodiments, the percentage of nicotine in the nicotine formulation is about 2%. According to some embodiments, the percentage of nicotine in the nicotine formulation is about 3%. According to some embodiments, the percentage of nicotine in the nicotine formulation is about 4%.

As used herein, when relating to nicotine percentages in liquid compositions, unless specified otherwise, the volume ratio, or w/w % is referred. For example, the phrase “the percentage of nicotine is within the range of 0.5 to 2%” refers to a liquid solution, in which a single weight unit of the solution includes from 0.005 to 0.02 the weight unit of nicotine. Specifically, adding 1 gr of nicotine to 99 gr of water will result in a 100 ml solution of 1% nicotine.

According to some embodiments, the concentration of nicotine in the formulation is within the range of 2 to 200 mg/ml. According to some embodiments, the concentration of nicotine in the formulation is within the range of 3 to 90 mg/ml. According to some embodiments, the concentration of nicotine in the formulation is within the range of 4 to 75 mg/ml. According to some embodiments, the concentration of nicotine in the formulation is within the range of 7.5 to 60 mg/ml. According to some embodiments, the concentration of nicotine in the formulation is within the range of 10 to 40 mg/ml. According to some embodiments, the concentration of nicotine in the formulation is about 10 mg/ml. According to some embodiments, the concentration of nicotine in the formulation is about 20 mg/ml. According to some embodiments, the concentration of nicotine in the formulation is about 30 mg/ml. According to some embodiments, the concentration of nicotine in the formulation is about 40 mg/ml.

According to some embodiments, the formulation is having a pH in the range of about pH 3 to about pH 5. According to some embodiments, the formulation is having a pH in the range of about pH 3.5 to about pH 4.5. According to some embodiments, the formulation is having a pH of about 4.

As used herein, the term “about” refers to a range of values ±20%, or ±10% of a specified value. For example, the phrase “having a pH of about 4” includes ±20% of 4, or from pH=3.2 to pH=4.8, or ±10% of 4, or from pH=3.6 to pH=4.4.

According to some embodiments, the nicotine formulation is an aqueous nicotine formulation. According to some embodiments, the buffer is an aqueous buffer.

According to some embodiments, the nicotine formulation comprises water. According to some embodiments, the buffer comprises water.

According to some embodiments, the buffer is useful within a pH range of about pH 3 to about pH 5. According to some embodiments, the buffer maintains the pH of a solution in range of about pH 3 to about pH 5. According to some embodiments, the buffer is approved for use in inhaling solutions.

According to some embodiments, the buffer is selected from the group consisting of citrate buffers, acetate buffers and phosphate buffers. According to some embodiments, the buffer is a citrate buffer. According to some embodiments, the buffer is a phosphate buffer.

According to some embodiments, the formulation further comprises a sweetener. According to some embodiments, the sweetener is selected from the group of artificial sweeteners including saccharine, aspartame, dextrose and fructose.

According to some embodiments, the formulation comprises nicotine as the only active ingredient.

The term “active ingredient” refers to an agent, active ingredient compound or other substance, or compositions and mixture thereof that provide some pharmacological and or biological, often beneficial, effect. Reference to a specific active ingredient shall include where appropriate the active ingredient and it's pharmaceutically acceptable salts.

According to some embodiments, the formulation further comprises at least one anti-coughing agent.

The term “anti-coughing agent” as used herein refers to an active agent used for the suppression, alleviation or prevention of coughing and irritations and other inconveniences in the large breathing passages that can, or may, generate coughing. Anti-coughing agent include, but are not limited to antitussives, which are used for which suppress coughing, and expectorants, which alleviate coughing, while enhancing the production of mucus and phlegm. Anti-coughing agents may ease the administration of inhaled aerosols.

According to some embodiments, the at least one anti-coughing agent is selected from expectorants, antitussives or both. According to some embodiments, the at least one anti-coughing agent is selected from the group consisting of menthol, dextromethorphan, dextromethorphan hydrobromide, hydrocodone, caramiphen dextrorphan, 3-methoxymorphinan or morphinan-3-ol, carbetapentane, codeine, acetylcysteine and combinations thereof.

According to some embodiments, the formulation further comprises at least one preservative. According to some embodiments, the preservative is selected from the group consisting of benzyl alcohol, propylparaben, methylparaben, benzalkonium chloride, phenylethyl alcohol, chlorobutanol, potassium sorbate, phenol, m-cresol, o-cresol, p-cresol, chlorocresol and combinations thereof.

According to some embodiments, there is provided a nebulizer comprising a porous medium configured to produce aerosols, a displaceable wetting mechanism configured to spread a liquid over the porous medium thereby to wet the porous medium and a gas channel configured to introduce pressure gradient to the porous medium, wherein the liquid comprises a nicotine formulation, comprising nicotine and a buffer, having a pH below 6

According to some embodiments, there is provided an aerosol comprising a nicotine formulation, comprising nicotine and a buffer, having a pH below 6.

It was surprisingly found that nebulization of a formulation as disclosed herein, namely, a formulation having a pH below 6, comprising nicotine and a buffer, results in droplets having a mass median aerodynamic diameter (MMAD) sufficiently small so as to reach the lungs, rather than precipitate on their way thereto. The small droplets reaching the lungs enable efficient respiratory delivery of the nicotine therapeutic agent. This is an overall advantage as maximizing the delivery of nicotine to the lungs, while minimizing its deposition in the mouth and throat are important in treating diseases or disorders related to the respiratory system.

The terms ‘droplet size’ and ‘mass median aerodynamic diameter’, also known as MMAD, as used herein are interchangeable. MMAD is commonly considered as the median particle diameter by mass. MMAD may be evaluated by plotting droplet size vs. the cumulative mass fraction (%) in the aerosol. MMAD may then be determined according to the interpolated droplet size corresponding to the point, where the cumulative mass fraction is 50%. This points represent the estimated values of particle sizes, above which the droplets are responsible to half to masses and below which the droplets are responsible to the other halves, in each solution.

According to some embodiments, there is provided an aerosol comprising a nicotine formulation, said aerosol comprising droplets having an MMAD of at most 10 microns, wherein the nicotine formulation comprises nicotine and a buffer, wherein the nicotine formulation is having a pH below 6, and wherein the nicotine is having a purity of at least 95%.

According to some embodiments, the aerosol comprises droplets having an MMAD within the range of 0.3 to 7 microns. According to some embodiments, the MMAD is within the range of 2 to 10 microns. According to some embodiments, the aerosol comprises droplets having an MMAD of less than 10 microns. According to some embodiments, the aerosol comprises droplets having an MMAD within the range of 0.3 to 7 microns. According to some embodiments, the MMAD is less than 5 microns.

According to some embodiments, the aerosol comprises droplets having a Geometric Standard Diameter (GSD) within the range of about 0.4-7 μm. According to some embodiments, the aerosol comprises droplets having a GSD within the range of about 2-5 (two to five) μm.

According to some embodiments, there is provided a nebulizer comprising a porous medium configured to produce aerosols, a liquid absorbing material configured to absorb a liquid, a wetting mechanism configured to press the liquid absorbing material against the porous medium, thereby to wet the porous medium with the liquid absorbed in the liquid absorbing material and a gas channel configured to introduce pressure gradient to the porous medium, wherein the liquid comprises a nicotine formulation, comprising nicotine and a buffer, wherein the nicotine formulation is having a pH below 6.

According to some embodiments, the porous medium is disposable.

According to some embodiments, the porous medium is in the form of a rod, a capsule or a flat disc.

The nebulizer disclosed herein may function as an inhaler under some circumstances. Thus, the terms ‘nebulizer’ and ‘inhaler’ as used herein may be interchangeable.

As used herein the term “aerosol” or “aerosolized drug” refers to a suspension of solid or liquid particles in a gas. As used herein “aerosol” or “aerosolized drug” may be used generally to refer to a drug that has been vaporized, nebulized, or otherwise converted from a solid or liquid form to an inhalable form including suspended solid or liquid drug particles. According to some embodiments, the drug particles include nicotine particles.

According to some embodiments, the nicotine formulation is an aerosol devoid of propellants.

The term “propellants” as used herein refers to pharmacologically inert liquids with boiling points from about room temperature (25° C.) to about −25° C. which singly or in combination exert a high vapor pressure at room temperature.

According to some embodiments, the nicotine is the only active ingredient in the nicotine formulation. According to some embodiments, the nicotine is having a purity of at least 95%.

According to some embodiments, the percentage of nicotine in the formulation is within the range of 0.1 to 10%.

According to some embodiments, the buffer is an aqueous buffer comprising citrate buffer.

According to some embodiments, the buffer is an aqueous citrate buffer.

According to some embodiments, the nicotine formulation is having a pH of about 4.

Nicotine is available in several forms but the present application of the medical port and delivery method proposes benefits and alternatives to tobacco addiction without exposure to the carcinogens of the tobacco products.

According to some embodiments, the liquid absorbing material is a sponge, a tissue, a foam material, a fabric or any other material capable of fully or partially retrievably absorbing liquids, wherein the liquid comprises the nicotine formulation. Each possibility is a separate embodiment of the invention.

According to some embodiments, the liquid absorbing material is configured to enable small diameter droplets to pass through the structure thereof and to obstruct large diameter droplets from passing through the material thereof.

According to some embodiments, the liquid absorbing material is configured to filter the passage of droplets depending on their diameter, such that large diameter droplets are obstructed by the liquid absorbing material.

The terms ‘sponge’ and ‘liquid absorbing material’ as used herein refer to any material that is capable of incorporating, taking in, drawing in or soaking liquids, and upon applying physical pressure thereto, release a portion or the entire amount/volume of the absorbed liquid. The physical pressure may be achieved for example by pressing the material against a solid structure.

According to some embodiments, the liquid absorbing material is having two sides, wherein a first side is facing the wetting mechanism and a second side is facing the porous medium. According to some embodiments, the wetting mechanism is a movable solid medium facing the first side of the liquid absorbing material. According to some embodiments, the wetting mechanism is in close proximity to the first side of the liquid absorbing material. According to some embodiments, the welling mechanism is attached to the first side of the liquid absorbing material.

The term ‘attached to’ as used herein includes, but is not limited to, linked, bonded, glued, fastened and the like.

According to some embodiments, the porous medium is having two sides, wherein a first side is facing the liquid absorbing material and a second side is facing the gas channel. According to some embodiments, the first side of the porous medium is facing the liquid absorbing material and the gas channel. According to some embodiments, the liquid absorbing material and the porous medium are in close proximity. According to some embodiments, the first side of the liquid absorbing material and the first side of the porous medium are in close proximity.

Without being bound by any theory or mechanism, a pressure gradient at the porous medium reflects the presence of value difference between the pressure at the first side of the porous material and the pressure at the second side of the porous material, such that pressure values vary inside the volume of the porous medium. These values range from the pressure value at the first side to the pressure value at the second side of the porous medium.

According to some embodiments, the nebulizer is portable. According to some embodiments, the nebulizer is a hand held nebulizer.

According to some embodiments, the gas channel is a gas delivery channel configured to introduce pressure gradient to the porous medium. According to some embodiments, the gas channel is a gas delivery channel configured to introduce pressurized gas to the porous medium. According to some embodiments, the gas channel is a gas suction channel configured to introduce sub-pressurized gas to the porous medium.

The term “channel” as used herein is interchangeable with any one or more of the terms port, passage, opening, orifice, pipe and the like.

According to some embodiments, a pressurized gas container is configured to deliver pressurized gas through the gas channel to the porous medium and create an ultra-atmospheric pressure on one side of the porous medium, thereby induce a pressure gradient at the porous medium.

The term “pressurized gas” as used herein is interchangeable with the term ‘compressed gas’ and refers to gas under pressure above atmospheric pressure.

According to some embodiments, a vacuum container or sub-atmospheric pressure container is configured to suck gas through the gas channel and create a sub-atmospheric pressure on one side of the porous medium, thereby induce a pressure gradient within the porous medium.

According to some embodiments, the gas channel is connected to a gas source. According to some embodiments, the gas source is a mobile gas source, such as, a gas container. According to some embodiments, the gas source is a gas pump, configured to introduce pressure gradient in the porous medium by pumping gas to or from the gas delivery channel. According to some embodiments, the gas source is a pressurized gas container, configured to contain pressurized gas and to induce a pressure gradient in the porous medium by releasing pressurized gas to the pressurized-gas delivery channel.

According to some embodiments, the nebulizer further comprises an opening configured to deliver the aerosols to a respiratory system of a subject. According to some embodiments, the opening is connected to a nozzle. According to some embodiments, the opening is mechanically connected to a nozzle. According to some embodiments, the nozzle is detachable.

The terms ‘nozzle’ and ‘outlet’ as used herein are interchangeable.

According to some embodiments, the wetting mechanism is a mechanic mechanism configured to apply pressure onto the liquid absorbing medium. According to some embodiments, the wetting mechanism is a pneumatic mechanism configured to apply pressure onto the liquid absorbing medium. In some embodiment the wetting mechanism is coupled with an actuator. According to some embodiments, the wetting mechanism comprises a metering pump adapted to delivering a predetermined volume of liquid at desired pressure(s) directly to the surface of the porous medium.

According to some embodiments, the nebulizer is mobile. According to some embodiments, the nebulizer is portable. According to some embodiments, the nebulizer is handheld. According to some embodiments, the nebulizer is powered by a mobile power source.

There is provided, according to some embodiments, a nebulizer housing configured to host at least one cartridge having a liquid absorbing material. The housing may further include any one or more of a porous medium, an opening, a nozzle connected to the opening, one or more container containing liquids, pharmaceutically active agents and composition comprising same, and a combination thereof.

According to some embodiments, the nebulizer housing is mobile. According to some embodiments, the housing is handheld. According to some embodiments, the nebulizer is powered by a mobile power source. According to some embodiments, the cartridge is disposable. According to some embodiments, the cartridge is recyclable. According to some embodiments, the liquid absorbing material is disposable. According to some embodiments, the cartridge is reusable.

According to some embodiments, the nebulizer is configured to communicate wirelessly with servers, databases, personal devices (computers, mobile phones) among others.

According to some embodiments, the nebulizer is assembled by introducing a cartridge into the housing.

There is provided, according to some embodiments, a nebulizer system comprising a housing, an opening in the housing configured to deliver an aerosols to a subject, a receptacle configured to receive a cartridge (the cartridge comprises a liquid absorbing material, and a porous medium, having at least one porous surface, configured to produce aerosols and a wetting mechanism configured to press the liquid absorbing material against the porous medium or against a surface of the porous medium), an actuator configured to control the wetting mechanism and a gas channel, to introduce a pressure gradient to the porous medium.

According to some embodiments, there is provided a nebulizer system comprising a receptacle configured to receive a cartridge. In combination, the nebulizer housing and the cartridge comprise the following elements: a liquid absorbing material, a porous medium having a porous surface, a wetting mechanism and at least one liquid or medication container.

The elements above may be comprised within the housing or the cartridge in various combinations; some examples of these combinations are given below for exemplary purposes, without limiting the disclosure from other possible combinations.

According to some embodiments, the housing comprises a receptacle, a porous medium, a liquid or medication container and a wetting mechanism, while the cartridge comprises a liquid absorbing material.

According to some embodiments, the housing comprises a receptacle, a porous medium and a liquid or medication container, while the cartridge comprises a liquid absorbing material and a wetting mechanism.

According to some embodiments, the housing comprises a receptacle and a liquid or medication container, while the cartridge comprises a porous medium, a liquid absorbing material and a wetting mechanism.

According to some embodiments, the housing comprises a receptacle and a porous medium, while the cartridge comprises a liquid or medication container, a liquid absorbing material and a wetting mechanism.

According to some embodiments, the housing comprises a receptacle while the cartridge comprises a liquid or medication container, a liquid absorbing material a porous medium, and a wetting mechanism.

According to some embodiments, the housing comprises at least two receptacles, a first receptacle configured to receiving a cartridge comprising a liquid absorbing material, and a second receptacle configured to receive a liquid or medication container.

According to some embodiments, the liquid absorbing material is presoaked with medication. According to some embodiments, the presoaked liquid absorbing material is hermetically or semi hermetically sealed. According to some embodiments, the seal is configured to be disrupted or otherwise removed upon usage. According to some embodiments, the seal is configured to be automatically disrupted or otherwise removed, for example, by an actuator in the nebulizer system. According to some embodiments, the seal is configured to be manually removed or disrupted by a user prior to use thereof.

According to some embodiments, the nebulizer system further comprises control mechanism configured to control the release of the liquid from the container containing same, into the liquid absorbing material. According to some embodiments, the control mechanism is configured to control the release of the liquid in a slow and/or gradual release manner. According to some embodiments, the nebulizer system further comprises deployment mechanism configured to deploy the medication or liquid from the container containing same and into the liquid absorbing material.

According to some embodiments, the nebulizer system or cartridge comprises a medication preparation mechanism for mixing the medication with a liquid to enable reconstitution of the medication, or dilution thereof, prior to aerosolization of the composition.

According to some embodiments, some mechanisms of the nebulizer system are configured to provide homogeneous or semi homogeneous wetting of the porous medium. According to some embodiments, the mechanisms are other than the liquid absorbing material and the wetting mechanism. Examples for such mechanisms include, but are not limited to, spray mechanism, wiping mechanisms and the like.

According to some embodiments, there is provided a nebulizer cartridge comprising a porous medium, wherein the porous medium comprises a plurality of pores, and wherein at least some of said plurality of pores comprise liquid comprising a nicotine formulation comprising nicotine and a buffer, wherein the nicotine formulation is having a pH below 6.

As used herein, “respiratory system” refers to the system of organs in the body responsible for the intake of oxygen and the expiration of carbon dioxide. The system generally includes all the air passages from the nose to the pulmonary alveoli. In mammals it is generally considered to include the lungs, bronchi, bronchioles, trachea, nasal passages, and diaphragm. For purposes of the present disclosure, delivery of a drug to the “respiratory system” indicates that a drug is delivered to one or more of the air passages of the respiratory system, in particular to the lungs.

The correlation between droplet size and deposition thereof in the respiratory tract has been established. Droplets around 10 micron in diameter are suitable for deposition in the oropharynx and the nasal area; droplets around 2-4 micron in diameter are suitable for deposition in the central airways and may be especially beneficial for delivery of nicotine the subjects in a need thereof.

According to some embodiments, the nicotine formulation may be included within a pharmaceutical composition. According to some embodiments, the pharmaceutical composition may comprise one or more pharmaceutically active agents, other than nicotine. According to some embodiments, the one or more pharmaceutically active agents are suitable or may be adjusted for inhalation. According to some embodiments, the one or more pharmaceutically active agents are directed for treatment of a medical condition through inhalation According to some embodiments, the at least one pharmaceutical composition comprise a nicotine formulation comprising nicotine and a buffer, wherein the nicotine formulation is having a pH below 6, wherein the nicotine is having a purity of at least 95%.

As used herein, a “pharmaceutical composition” refers to a preparation of a composition comprising one or more pharmaceutically active agents, such as nicotine, suitable for administration to a patient via the respiratory system.

According to some embodiments, the pharmaceutical composition further comprises at least one pharmaceutical acceptable carrier. In other embodiments, the pharmaceutical composition may further comprise one or more stabilizers.

According to some embodiments, the nebulizer provides an aerosol containing a therapeutically effective amount of the nicotine formulation.

As used herein, the term “therapeutically effective amount” refers to a pharmaceutically acceptable amount of the nicotine formulation which prevents or ameliorates, at least partially, the symptoms signs of a particular disease or disorder, for example nicotine withdrawal symptoms, in a living organism to whom it is administered over some period of time.

According to some embodiments, the nebulizer provides an aerosol containing an effective amount of nicotine.

As used herein, the term “effective amount of nicotine” refers to an amount of a nicotine, which is in the range of nicotine amount absorbed upon smoking a cigarette.

As exemplified herein (e.g. Examples 4 and 5) the nebulizer and formulation of the invention provide an effective dose of nicotine, which is comparable to the amount of nicotine delivered through the lungs, by smoking a cigarette. Without wishing to be bound by any theory or mechanism of action, the high dosage of nicotine that reaches the lungs by inhaling the nicotine formulation using the nebulizer disclosed herein is attributed to the small aerosol droplets, having MMAD within the range of about 2.5 to 3 microns. It is noted that such small droplets were maintained even at aerosol produced with high nicotine concentrations, of about 4%. Thus, high nicotine concentrations can be inhaled and reach the lungs using the nebulizer and nicotine formulations disclosed herein. In contrast, it seems that solutions having pH of about 7 to 8, result in aerosols having larger droplets which typically hinder the delivery of solutes to the lungs, thereby leading to low dosages that cannot provide the desired therapeutic effect.

According to some embodiments, there is provided a method for treating a disease or disorder in a subject in need thereof comprising administering, via inhalation, to the subject an aerosol comprising a nicotine formulation, comprising nicotine and a buffer, wherein the nicotine formulation is having a pH below 6.

According to some embodiments, the disorder is nicotine withdrawal syndrome.

According to some embodiments, the subject is having a respiratory disease or disorder. According to some embodiments, the respiratory disease or disorder is a pulmonary disease.

According to some embodiments, the disease is selected from the group consisting of asthma, bronchitis, emphysema, lung infection, cystic fibrosis, AAT deficiency, COPD, ARDS, IRDS, BPD, and MAS. Each possibility is a separate embodiment of the invention.

According to some embodiments, the subject is having a respiratory disease affecting the air ways, the alveoli or the interstitium, such as, asthma, chronic obstructive pulmonary disease, chronic bronchitis, emphysema, acute bronchitis, cystic fibrosis, pneumonia, tuberculosis, fragile connections between alveoli, pulmonary edema, lung cancer in its many forms, acute respiratory distress syndrome, pneumoconiosis, mouth and pharynx cancer, tracheal tumors and interstitial lung disease among others.

### EXAMPLES

### Example 1: Preparation of Formulation for Inhalation

The formulation solution used in the experiments below included nicotine in a citrate buffer adjusted to pH=4. The solution was prepared by combining 1.5 ml nicotine (ca. 1.5 gr) with citrate buffer (0.1 M) and 6M hydrochloric acid until pH=4 was reached (1.62 ml), and adding water for injection to a final volume of 150 ml. The amount of nicotine in the formulation is about 1.5 gr in 150 ml solution, or 10 mg/ml nicotine. Similarly, formulations for inhalation having 20 mg/ml nicotine, 30 mg/ml nicotine and 40 mg/ml nicotine, were prepared according to the above procedure, using 3 gr nicotine, 4.5 gr nicotine and 6 gr nicotine, respectively, in 150 ml solutions. The volume fractions of nicotine are about 1% v/v, 2% v/v, 3% v/v, and 4% for the formulations having 10 mg/ml, 20 mg/ml, 30 mg/ml and 40 mg/ml nicotine, respectively.

### Example 2: Formulation Specification Test

Analytical methods were applied to characterize the various formulations. For example, a formulation having a volume fractions of nicotine of about 1% v/v (corresponding to 10 mg/ml exhibited colorless-yellowish color where IR spectrum thereof was found consistent with standard IR spectrum of nicotine.

The exemplary formulation solution was checked for presence of the related substances cotinine, myosmine and nicotine-N-oxide by HPLC. It was found that the solution contains less than 1% cotinine, less than 1% myosmine and less than 3% nicotine-N-oxide.

### Example 3: Mass Distribution on Impactor Parts

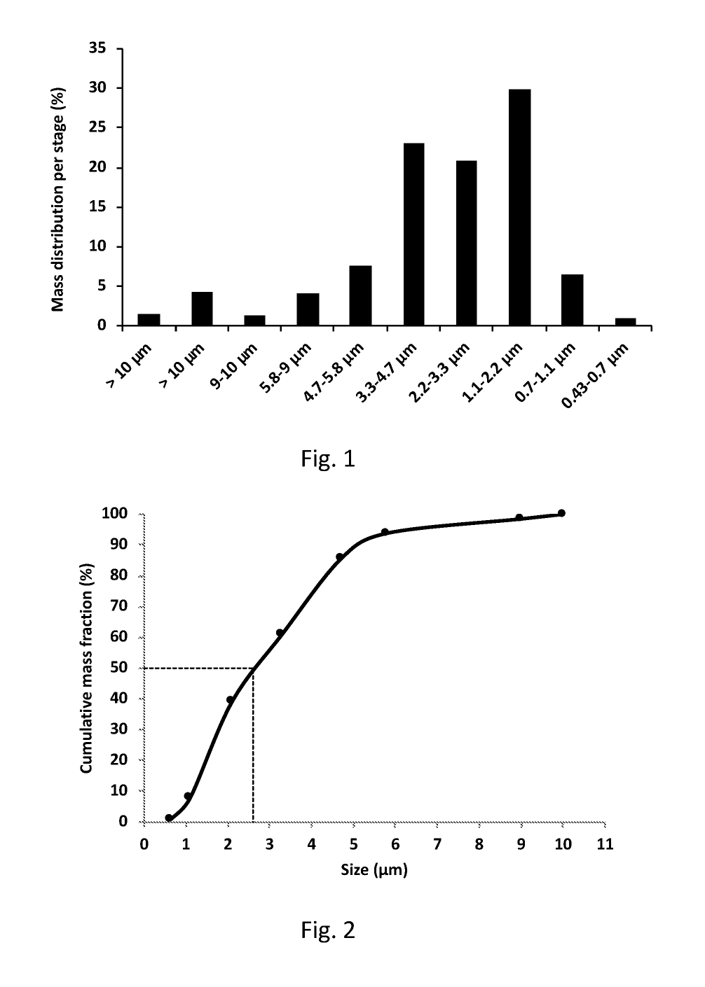

Particle size distribution testing was conducted using cascade impactor validated method with the 10 mg/ml nicotine formulation of Example 1. The limits for the median diameter range from 1.5 to 3 micron and the limit on the sub 5 micron particles/droplets was set at 70%. The results are presented in FIG. 1 and relate to the formulation nebulized with a nebulizer as disclosed herein, which comprises a porous medium and a displaceable wetting mechanism.

Relative mass of the nebulized solution was measured against its particle size, which was measured between 0.43 micrometers and over 10 micrometers.

FIG. 1 is a chart representing Mass Distribution on Impactor parts in an aerosol depicting the relative mass of the aerosol in each particle diameter size group, where the particle diameter groups are 0.43 to 0.7 microns; 0.7 to 1:1 microns; 1.1 to 2.2 microns; 2.2 to 3.3 microns; 3.3 to 4.7 microns; 4.7 to 5.8 microns; 5.8 to 9 microns; and over 10 microns.

As can be seen in FIG. 1, the majority of aerosol mass was provided in droplets having diameters in the range of 1.1 to 4.7 microns. More specifically, droplets in the range of 1.1 to 2.2 microns accounted for about 30% of the total aerosol mass; droplets in the range of 2.2 to 3.3 microns accounted for about 21% of the total aerosol mass; and droplets in the range of 3.3 to 4.7 microns accounted for about 23% of the total aerosol mass. In total, droplets in the range of 1.1 to 4.7 microns amounted to about 74% of the total aerosol mass, whereas droplets having diameters of more than 5.8 microns or less than 0.7 microns contributed only very small amounts of aerosol.

Table 1 lists results droplet size distribution results from four separate experiments. The results are categorized in fine particle fractions: below 1 micron; below 3 microns; below 5 microns; and a fraction of above 5 microns. The mean value, 95% CI, Standard Deviation and % RSD are also incorporated. It is apparent from Table 1 that more than half of the total mass of the aerosol was delivered in droplets having diameters up to 3 microns. Moreover, only 20% of the aerosol mass was delivered in large droplets of more than 5 microns. Very small droplets were also accountable for small amounts of the delivered aerosol mass, when only below 6% of the mass was delivered in droplets of up to 1 micron in diameter.

TABLE 1
Size distribution of aerosols containing the nicotine formulation

Distribution of delivered mass (%)

| Replicate | <1 μm | <3 μm | <5 μm | >5 μm |
|---|---|---|---|---|
| 1 | 7.32 | 52.81 | 78.18 | 21.82 |
| 2 | 5.35 | 53.3 | 80.8 | 19.2 |
| 3 | 3.96 | 50.3 | 79.59 | 20.41 |
| 4 | 6.33 | 56.78 | 83.16 | 16.84 |
| Mean | 5.74 | 53.3 | 80.44 | 19.56 |
| 95% CI | 1.22 | 2.26 | 1.79 | 1.79 |
| Standard Deviation | 1.24 | 2.31 | 1.83 | 1.83 |
| % RSD | 21.64 | 4.34 | 2.27 | 9.34 |

Table 2 lists results of mass median aerodynamic diameter (MMAD) and geometric standard deviation (GSD) in four separate experiments plotting the delivered mass as a function of the droplet diameter. The mean value, 95% CI, Standard Deviation and % RSD are also incorporated. Table 2 shows that the MMAD of the particles was 2.51 microns, which is also in accordance with the results depicted in Table 1, which shows that the majority of aerosol mass was concentrated in droplets having diameters between 1 and 3 microns.

TABLE 2
MMAD and GSD of aerosols containing the nicotine formulation

| Replicate | MMAD (μm) | GSD |
|---|---|---|
| 1 | 2.4 | 1.84 |
| 2 | 2.54 | 1.77 |
| 3 | 2.68 | 1.74 |
| 4 | 2.42 | 1.77 |
| Mean | 2.51 | 1.78 |
| 95% CI | 0.11 | 0.03 |
| Standard Deviation | 0.11 | 0.03 |
| % RSD | 4.43 | 1.9 |

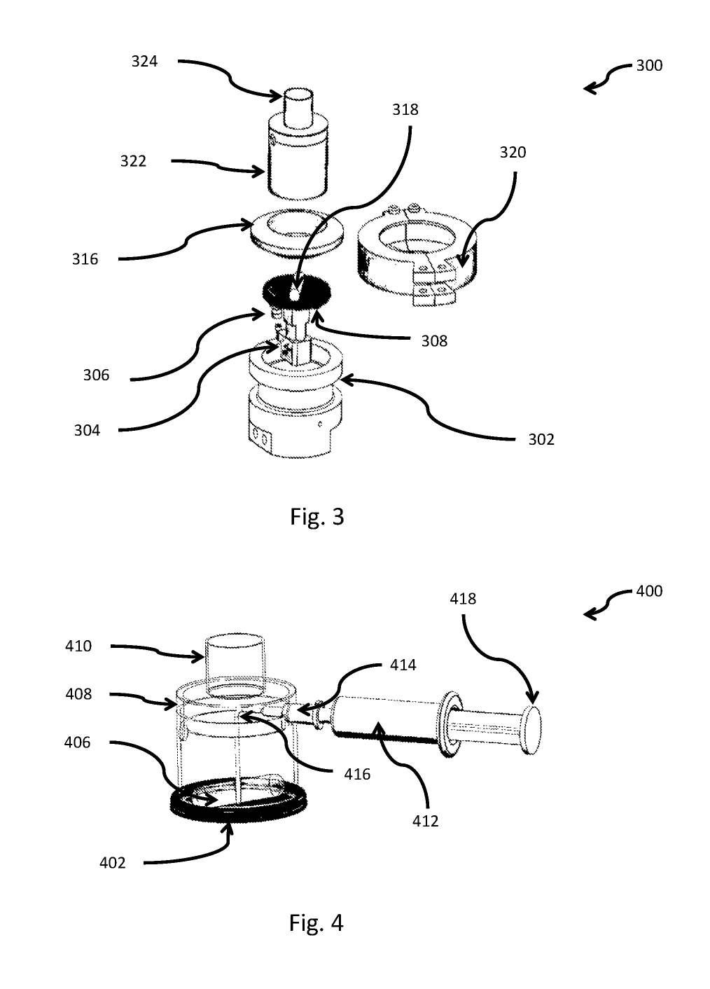

Finally, FIG. 2 is a chart representing cumulative Mass Distribution of the aerosol in the experiment. It depicts the cumulative mass fraction vs. the droplet size in micrometers. The dotted line represents the estimated value of particle size, above which the droplets are responsible to half to mass and below which the droplets are responsible to the other half. Again, it is seen that half of the mass was delivered in droplets having diameters below 2.6 microns. Also, droplets having diameters below 1.25 microns and droplets having diameters above 5 microns accounted for only very small amounts of aerosol.

### Example 4: Pharmacokinetics—Consumer-Goods Trial

An open label consumer product investigation in five healthy male volunteers was performed to determine the performance and tolerability of aerosol produced with the nebulizer, according to some embodiments.

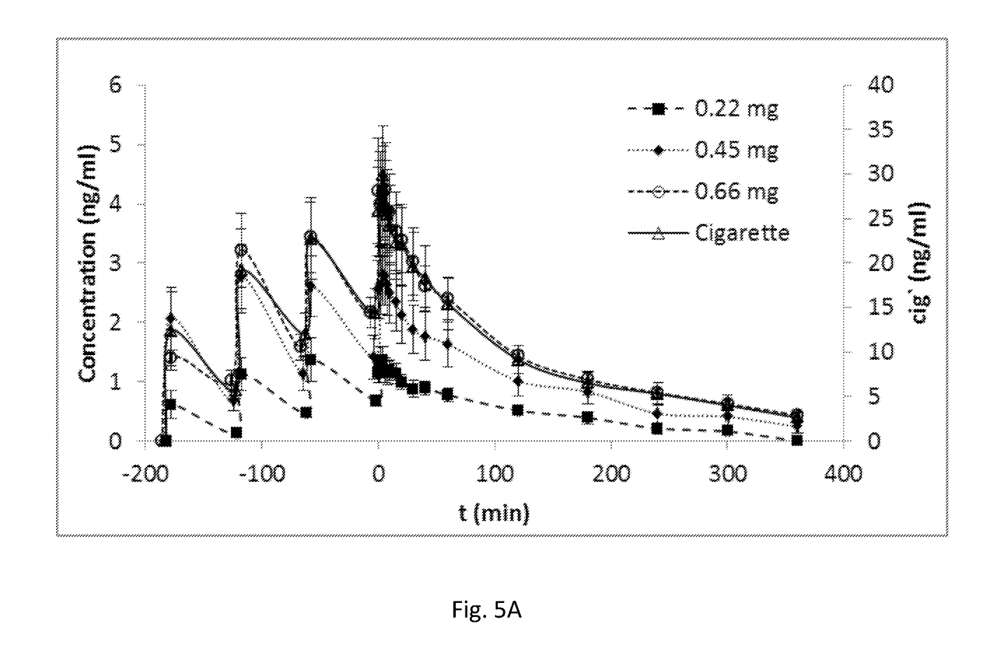

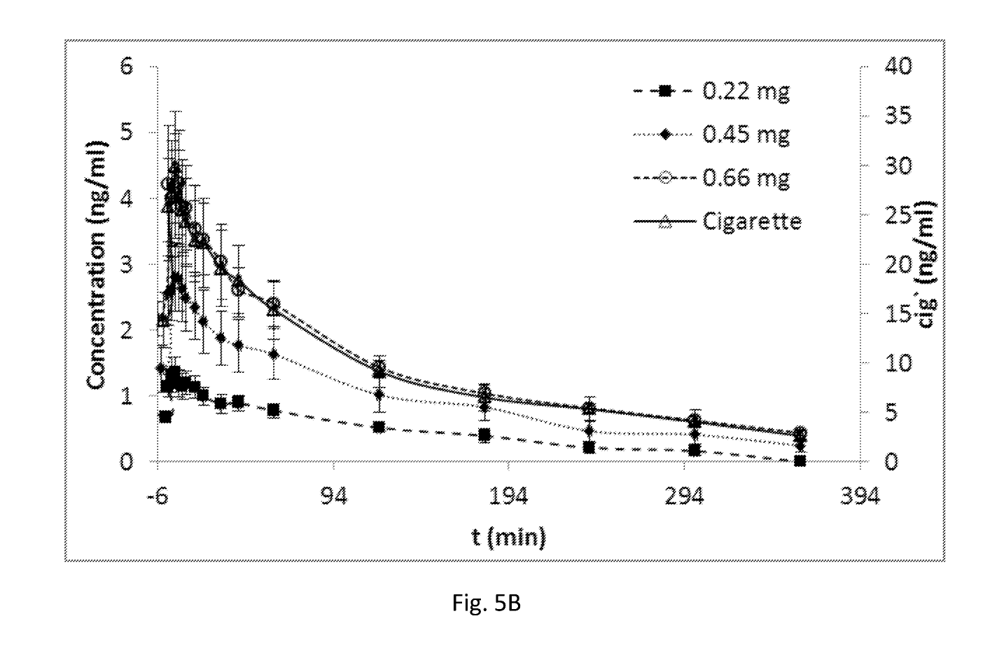

In brief, the nebulizer used in the current study was a desktop device that functions according to drop-on-demand mechanism. The nebulizer housed modular disposable capsule(s), such as shown in FIGS. 3 and 4, each functioning as porous disk 308 and disposable containers (in the form of glass syringes 412) holding the nicotine formulation. The capsule further contained Teflon coated magnet 318 and housing 322.

Aerosol generation was not triggered by forceful dispensing of liquid through the porous membrane, but rather by driving air through a wetted porous medium.

Reference is now made to FIG. 3, which constitutes an exploded view of the components of a modular capsule 308 and stirring motor assembly 300 of the nebulizer used in the consumer-goods trial. The stirring motor is part of the desktop unit and served to induce rotation in the capsule's magnet. The stirring motor 304 is part of the desktop unit and served to induce rotation in the Teflon coated magnet 318. The stirring motor 304, placed on top of a stirring motor base 306, is nested inside a stainless steel housing that receives pressurized air and also used as housing for the porous medium 308. The aerosol producing capsule 308 may be connected and disconnected from the stirring motor 304 housing by means of a capsule top flange 316 and capsule locking clamp 320. The stirring motor housing (not shown) becomes pressurized only when a capsule, including the capsule housing 322, is connected and fastened thereto and pressurized air is supplied. Pressure builds up due to the resistance to flow posed by the porous medium 308. The pressure level is dictated by the porosity of the porous disk and the volumetric air flow driven therethrough. The Teflon coated magnet 318 in the capsule is induced to rotate by the rotating magnets of the stirring motor 306 and served to evenly spread the delivered liquid across the surface of the porous medium 308. The assembly further includes a mouthpiece 324.

Reference is now made to FIG. 4, which constitutes a perspective view of the pressurized core 400 of the nebulizer used in the consumer-goods trial. The modular capsule is comprised of a porous medium 402, stainless steel capsule tube 408, stainless steel mouthpiece adapter 410, Teflon coated magnet 406, and bent stainless steel needle 414 with flexible silicone tubing 416. A prefilled syringe 412 is connected to the capsule and predetermined volumes of liquid forced out by virtue of mechanical pressure exerted on the syringe plunger 418 via a step motor (not shown). The liquid travels from the syringe 412 via the bent needle 414 and silicone tubing 416 to the surface of the porous medium 402. The rotation of the magnet 406 serves to spread the liquid across the surface of the porous, medium 402, enabling aerosolization of the liquid at high yield.

During the consumer-goods trial, pressurized air from an external compressor (not illustrated) was fed via the desktop unit and through the porous medium 402. As the surface of the porous medium 402 was wetted aerosol was produced. Predetermined amounts of liquid were transferred from the syringe 412 to the capsule upon user actuation. User actuation may be set as either breath actuated or mechanically actuated. In the breath actuation mode the reduction in pressure triggered by the user inhalation was sensed by the differential pressure sensor (not illustrated). The differential pressure sensor is connected via tubing to the capsule and can thus sense changes in pressure. The differential pressure sensor is in communication with a micro-processor which activates a step motor (not illustrated). The step motor is connected to a lever that applies pressure to the plunger of the syringe 418. The length of the step was chosen so as to result in the dispensing of a 4-6.5 microliter drop from the syringe.

The step motor was controlled by an integrated circuit and software. The length of a single step can be changed by reprograming yet cannot be set by the user. User actuation (breath or mechanical) sensed by the different sensors and the information was conveyed to the microprocessor. Actuation triggered a single step of the step motor. This resulted in the transfer of a fixed amount of liquid from the syringe to the surface of the porous medium (e.g. disc). Typically, this volume was within the range of 4-6.5 microliters and was completely aerosolized in about 5 seconds. Once actuated the system is indifferent to any additional actuations for a period of 6 seconds. This temporary insensitivity to additional actuations was designed to prevent accumulation of a surplus of liquid on the surface of the porous disk. This quality assurance mechanism guarantees that aerosolization occurs in a series of well-defined steps.

The nicotine formulation was administered via inhalation using the nebulizer at three dose levels following an ascending dose protocol. Subjects were familiarized with the aerosol and the nebulizer using a placebo formulation—saline (0.9% NaCl aqueous solution)—on the day prior to initiation of the study, i.e. prior to receiving the first dose of the active product.

Subjects received the product at the following three dose levels on three sequential days: 0.22 mg on each cycle in Day 1, 0.45 mg on each cycle in Day 2, and 0.67 mg on each cycle in Day 3. On Day 4 subjects smoked cigarettes. For each cycle of inhalations, in Day 1-Day 3, subjects were instructed to inhale the aerosol from the mouthpiece of the nebulizer for 4 seconds. During the study, four (4) cycles of inhalation schedule were performed daily. The time between daily cycles was one hour. Each cycle included a specific number of inhalations (6, 13 or 19), with 15 seconds between two consecutive inhalations, where each cycle was repeated 4 times a day, at four consecutive hours. The daily cycles were as follows:

Day 1 (0.22 mg)—6 inhalations per cycle;

Day 2 (0.45 mg)—13 inhalations per cycle;

Day 3 (0.67 mg)—19 inhalations per cycles; and

Day 4—smoking a cigarette containing about 1 mg Nicotine, through 10 inhalations, each lasting about 2 seconds.

Throughout the study, individual treatments are administered in dedicated rooms with continuous air extraction, such that a single subject is dosed in each room at a given time with at least 10 minutes clean air time between dosing occasions.

On each day of the experiment, blood samples were drawn as follows:
(i) for the first three (3) hourly inhalations/smoking: immediately before inhalation of the nicotine formulation and 2 minutes thereafter;
(ii) on the 4th inhalation (or smoking, on Day 4) of each day (i.e. 4th hour), blood samples were collected at 0 min. (pre inhalation/smoking), immediately after the last inhalation/smoking and at 2, 4, 6, 8, 10, 15, 20, 30, 40, 60, 120, 180, 240, 300 and 360 mins after the last inhalation/smoking.

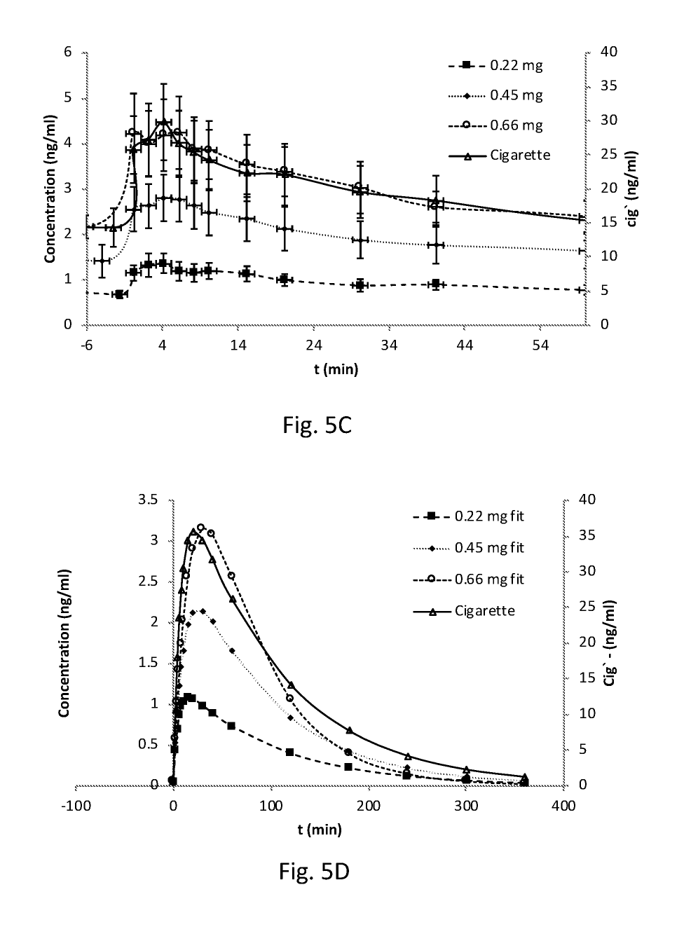

FIG. 5A shows average nicotine plasma concentrations over time, per day/dose (or cigarette smoking), for all four daily cycles over the 4 days of the experiment. Results are shown with standard deviations. Left vertical axis represent concentration scale for all inhaled doses on Days 1-3, while right vertical axis (cig′) represents concentration scale corresponding to cigarette smoking on Day 4. Peaks represent nicotine concentrations in the blood after each hourly inhalation cycle. Analyses of maximal concentration and time, Cmax and Tmax, respectively, for each daily dose were derived from the pharmacokinetics data collected before and after the 4th daily cycle (FIG. 5B). Time zero (t₀) refers to the beginning of the last inhalation (4th) inhalation cycle. Time is given in minutes where all time values prior to t₀ were given negative values.

FIG. 5B shows average nicotine plasma concentration versus time of the 4th daily dose for each treatment.

FIG. 5C shows a mathematical best fit to the average values given in FIG. 4.

Table 3 summarizes the Cmax and Tmax values calculated based on the 4th daily run for the different treatments.

TABLE 3
Data summary table of Cmax and Tmax.

| Dose | | Mean | SD | Min | Max | Median |
|---|---|---|---|---|---|---|
| 0.22 mg | Cmax (ng/ml) | 1.43 | 0.53 | 0.69 | 1.95 | 1.35 |
| | Tmax (min) | 4.17 | 1.41 | 2.17 | 6.17 | 4.17 |
| 0.45 mg | Cmax (ng/ml) | 2.91 | 1.13 | 1.34 | 3.95 | 3.55 |
| | Tmax (min) | 4.97 | 1.10 | 4.17 | 6.17 | 4.17 |
| 0.66 mg | Cmax (ng/ml) | 4.49 | 1.99 | 2.80 | 7.01 | 3.41 |
| | Tmax (min) | 5.77 | 3.58 | 0.17 | 10.17 | 6.17 |
| Cigarette | Cmax (ng/ml) | 29.83 | 12.56 | 10.3 | 40.86 | 36.32 |
| | Tmax (min) | 4.17 | 0.00 | 4.17 | 4.17 | 4.17 |

The results indicate that average Tmax values for all tested doses are in the range of 1-6 min., which is similar to Tmax values of cigarettes. Moreover, it is apparent from the results that Cmax is highly proportional to the inhaled dose, allowing to easily extrapolate from the data disclosed herein the dose required achieve any given Cmax values, including Cmax similar to the Cmax of cigarettes, which is typically within the range of 20-30 ng/ml. For example, based on the fact that average Cmax is 1.43 ng/ml corresponds to inhalation of 0.22 mg, and 4.49 ng/ml corresponds to inhalation of 0.66 mg, it can be extrapolated that inhalation of about 4-6 mg nicotine, using the formulation and inhaler disclosed herein will result in Cmax of about 20-30 ng/ml.

### Example 5: Users Experience—Clinical Trial

All patients participating in the open label study described in Example 4, above, were instructed to complete a questionnaire with regard to their satisfaction and other factors related to their impression and overall feeling during the study. The questionnaire included 10 questions as presented in Table 4. All of the rating were made on 10-points scale, where 10 indicates maximum satisfaction or the like and 1 indicates maximal dissatisfaction or the like.

Questionnaire results indicate the presence of a clear trend: higher ratings for higher inhaled dosages. Mean scores were highest for the 0.66 mg inhalation for 9 out of 10 questions. For comparison, regular cigarette smoking showed highest mean score only for a single question, while exhibiting the lowest mean scores for 7 out of 10 questions.

The rating provided by the users suggest that the inhaling the nicotine formulation of Example 1 through the nebulizer is advantageous over cigarette smoking in terms of user's experience, primarily for the dose of 0.66 mg. As shown in FIGS. 5A-5D and in Table 3, above, the pharmacokinetics of the dose of 0.66 mg is similar to that of cigarettes, which may explain the high rate of satisfaction.

TABLE 4
Questionnaire data

| Question | Dose | Mean | SD | Min | Max | Median |
|---|---|---|---|---|---|---|
| Felt Awake | 0.22 mg | 8.5 | 2.07 | 5 | 10 | 9.5 |
| After Using the | 0.45 mg | 8.5 | 2.07 | 5 | 10 | 9.5 |
| Product | 0.66 mg | 8.7 | 1.86 | 5 | 10 | 9 |
| | Cigarette | 7 | 1.9 | 5 | 10 | 7 |
| Found | 0.22 mg | 8.5 | 2.35 | 4 | 10 | 9.5 |
| Comfortable to | 0.45 mg | 8.8 | 1.33 | 7 | 10 | 9 |
| Inhale | 0.66 mg | 9 | 0.89 | 8 | 10 | 9 |
| | Cigarette | 8.5 | 2.07 | 5 | 10 | 9.5 |
| Found the | 0.22 mg | 9.7 | 0.52 | 9 | 10 | 10 |
| Device/Cigarette | 0.45 mg | 9.8 | 0.41 | 9 | 10 | 10 |
| Easy To Use | 0.66 mg | 10 | 0 | 10 | 10 | 10 |
| | Cigarette | 9.8 | 0.41 | 9 | 10 | 10 |
| Found the | 0.22 mg | 7.2 | 2.71 | 4 | 10 | 7.5 |
| Product | 0.45 mg | 8.2 | 2.14 | 5 | 10 | 9 |
| Relaxing | 0.66 mg | 8.5 | 1.87 | 5 | 10 | 9 |
| | Cigarette | 7.5 | 2.17 | 4 | 10 | 8 |
| Liked Using the | 0.22 mg | 7.7 | 2.07 | 5 | 10 | 7.5 |
| Device/Cigarette | 0.45 mg | 8 | 1.79 | 5 | 10 | 8.5 |
| | 0.66 mg | 8.3 | 1.86 | 5 | 10 | 8.5 |
| | Cigarette | 6 | 3.58 | 0 | 10 | 6 |
| The Product | 0.22 mg | 7.5 | 2.35 | 4 | 10 | 7.5 |
| Helped Me | 0.45 mg | 8 | 2.1 | 5 | 10 | 8.5 |
| Concentrate | 0.66 mg | 8.2 | 1.94 | 5 | 10 | 8.5 |
| | Cigarette | 7.5 | 2.43 | 5 | 10 | 7.5 |
| The Product | 0.22 mg | 8.3 | 1.86 | 5 | 10 | 8.5 |
| Made Me Feel | 0.45 mg | 8.2 | 1.94 | 5 | 10 | 8.5 |
| Less Irritable | 0.66 mg | 8.3 | 1.86 | 5 | 10 | 8.5 |
| | Cigarette | 8.5 | 1.87 | 5 | 10 | 9 |
| The Product | 0.22 mg | 7.5 | 3.15 | 3 | 10 | 9 |
| Tastes Good | 0.45 mg | 8 | 1.9 | 5 | 10 | 8 |
| | 0.66 mg | 8 | 2.1 | 4 | 10 | 8.5 |
| | Cigarette | 6.2 | 3.54 | 0 | 10 | 6.5 |
| The Product | 0.22 mg | 7.7 | 2.42 | 4 | 10 | 8 |
| Was Enjoyable | 0.45 mg | 8.2 | 1.83 | 5 | 10 | 8 |
| to Use | 0.66 mg | 8.8 | 1.94 | 5 | 10 | 9.3 |
| | Cigarette | 7.5 | 2.81 | 4 | 10 | 8 |
| The Product | 0.22 mg | 7.7 | 2.34 | 5 | 10 | 8 |
| Was Satisfying | 0.45 mg | 8.5 | 1.38 | 7 | 10 | 8.5 |
| | 0.66 mg | 8.7 | 1.63 | 6 | 10 | 9 |
| | Cigarette | 7.5 | 3.02 | 2 | 10 | 8 |

### Example 5: Pharmacokinetics of Commercial Products

The results presented in FIGS. 5A-D and in Table 3 show the pharmacokinetic profiles of the nicotine formulation disclosed herein, and highlight the fact that the formulations have pharmacokinetics which is similar to that of a cigarette. Specifically, mean Tmax for all tested doses was in the range of 4-6 minutes, similar to that of the cigarette and concentration profile over time is similar between all tested inhalation dosages and cigarettes. In addition, the high proportionality between inhaled dose and Cmax enables proper extrapolation of the expected inhalation dose required to derive any desired Cmax value, such as, a Cmax similar to that of a cigarette, i.e. in the range of 20-30 ng/ml.

In contrast, none of the common commercial products provide pharmacokinetics that is equivalent to that of cigarettes, as shown in Table 5.

TABLE 5
Pharmacokinetics of commercial products

| Product (nicotine contents) | Cmax (ng/ml) | Tmax (min) |
|---|---|---|
| Patches for transdermal delivery (21 mg) | 12 | 110 |
| Chewing gums (4 mg) | 10 | 30 |
| Nasal spray (1 mg) | 6 | 8 |
| Lozenges (2 mg) | 8 | 30 |
| Inhalers (4 mg) | 7 | 15 |

Table 5 exhibits the challenge that most of the current commercially available products face in achieving Tmax and Cmax within the range of a regular cigarette. The Tmax values of the common commercial products, such as those listed in Table 5 are much higher than Tmax of a regular cigarette. Moreover, Cmax of the common commercial products is lower than the average Cmax values achieved with cigarette smoking.

### Example 6: Mass Distribution as a Function of Nicotine Concentration

Testing of particle size distribution was conducted using cascade impactor validated method.

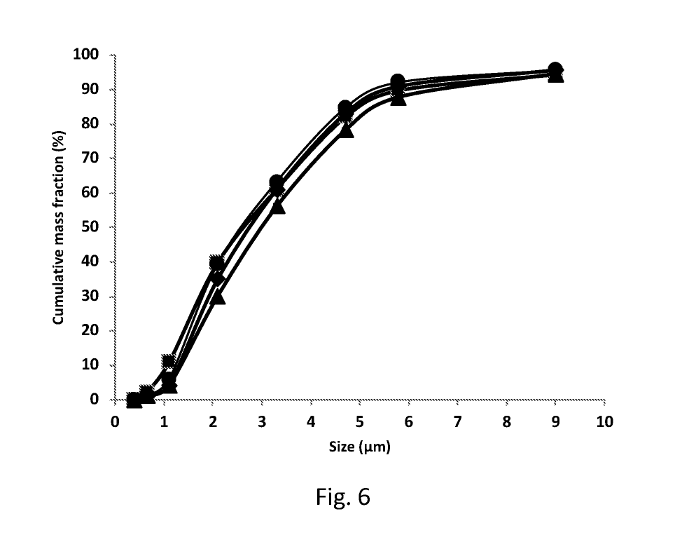

The results presented in FIG. 6 relate to solutions having formulation of 10-40 mg/ml nicotine as described in Example 1, nebulized with a nebulizer as disclosed herein.

Relative mass of the nebulized solution was measured against its particle size, which was measured between 0.43 micrometers and over 10 micrometers.

FIG. 6 is a chart representing cumulative Mass Distribution of the aerosol in the experiment. It depicts the cumulative mass fraction vs. the droplet size in micrometers for the four formulations comprising 10 mg/ml (squares), 20 mg/ml (circles), 30 mg/ml (diamonds) and 40 (triangles) mg/ml nicotine in citrate buffers adjusted to pH=4. The MMAD of each solution was measured as described above. The results indicate that increasing the concentration of nicotine in the formulations has a moderate effect on the MMAD of the resulting droplet, namely, MMAD increased from about 2.6 to about 3.0 microns, when the concentration quadrupled from 10 to 40 mg/ml. FIG. 6 also represents droplets having diameters below 1.25 microns and droplets having diameters above 5 microns, however, they account for a very small amounts of aerosol. The data suggests that the nicotine formulations disclosed herein, which encompass nicotine concentrations larger than 10 mg/ml may be applicable for delivering nicotine to the lungs, as they maintain small MMAD.

Although the invention is described in conjunction with specific embodiments thereof, it is evident that numerous alternatives, modifications and variations that are apparent to those skilled in the art may exist. It is to be understood that the invention is not necessarily limited in its application to the details of construction and the arrangement of the components and/or methods set forth herein. Other embodiments may be practiced, and an embodiment may be carried out in various ways. Accordingly, the invention embraces all such alternatives, modifications and variations that fall within the scope of the appended claims.

1.-35. (canceled)

36. An aqueous nicotine formulation comprising nicotine and a buffer, the formulation is having a pH within the range of 3.5 to 4.5, wherein the nicotine is having a purity of at least 95% and wherein the percentage of nicotine based on the total mass of the formulation is within the range of 0.5 to 8%.

37. The aqueous nicotine formulation of claim 36, wherein the buffer is a citrate buffer.

38. The aqueous nicotine formulation of claim 36, wherein said formulation is an aerosol having a pH of about 4.

39. The aqueous nicotine formulation of claim 36, wherein said formulation is an aerosol devoid of propellants.

40. The aqueous nicotine formulation of claim 36, wherein the concentration of nicotine in the formulation is within the range of 10 to 40 mg/ml.

41. The aqueous nicotine formulation of claim 36, having a pH of about 4.

42. The aqueous nicotine formulation of claim 36, further comprising a sweetener selected from the group consisting of saccharine, aspartame, dextrose and fructose.

43. The aqueous nicotine formulation of claim 36, further comprising at least one anti-coughing agent selected from expectorants, antitussives or both.

44. An aerosol comprising an aqueous nicotine formulation, said aerosol comprising droplets having an MMAD of at most 10 microns, wherein the aqueous nicotine formulation comprises nicotine and a buffer, having a pH within the range of 3.5 to 4.5, and wherein the nicotine is having a purity of at least 95%.

45. The aerosol of claim 44, comprising droplets having an MMAD within the range of 0.3 to 7 microns.

46. The aerosol of claim 44, comprising droplets having an MMAD is less than 5 microns.

47. The aerosol of claim 44, comprising droplets having droplets having a GSD within the range of 2-5 μm.

48. The aerosol claim 44, wherein the concentration of nicotine in the formulation is within the range of 10 to 40 mg/ml.

49. The aerosol of claim 44, wherein the aqueous nicotine formulation is having a pH of about 4.

50. The aerosol of claim 44, consisting essentially nicotine and the buffer, wherein the buffer is an aqueous buffer.

51. A method for treating a disease or disorder in a subject in need thereof comprising administering, via inhalation, to a subject in need thereof an aerosol comprising an aqueous nicotine formulation, comprising nicotine as the only active ingredient and a buffer, wherein the nicotine formulation is having a within the range of 3.5 to 4.5.

52. The method of claim 51, wherein the nicotine is having a purity of at least 95%.

53. The method of claim 51, for wherein the disorder is nicotine withdrawal syndrome.

54. The method of claim 51, wherein the subject is having a disease or disorder related to the respiratory system.
